(19) United States
(12) Patent Application Publication
(10) Pub. No.: US 2011/0039885 A1
(43) Pub. Date: Feb. 17, 2011

(54) METHODS FOR INCREASING ENDOTHELIAL PROGENITOR CELLS

(75) Inventors: Stephen J. Klaus, San Francisco, CA (US); Ingrid Langsetmo Parobok, Port Angeles, WA (US); Oksana Sirenko, San Carlos, CA (US)

Correspondence Address: FIBROGEN, INC., 409 Illinois Street, San Francisco, CA, 94158, US

(73) Assignee: FibroGen, Inc., San Francisco, CA (US)

(21) Appl. No.: 12/734,893

(22) PCT Filed: Dec. 8, 2008
(86) PCT No.: PCT/US2008/013515

§ 371 Date: Oct. 25, 2010

Related U.S. Application Data
(60) Provisional application No. 61/005,664, filed on Dec. 6, 2007.

(51) Int. Cl. A61K 31/47

(52) U.S. Cl. 514/307

(57) ABSTRACT

The present invention relates to methods and compounds useful for increasing endothelial progenitor cell levels in blood and bone marrow. Methods and compounds for increasing endothelial progenitor cell mobilization are also provided.

### FIELD OF THE INVENTION

The present invention relates to methods and compounds useful for increasing endothelial progenitor cell levels in blood and bone marrow. Methods and compounds for increasing endothelial progenitor cell mobilization are also provided.

### BACKGROUND

Endothelial progenitor cells (EPCs) are immature endothelial cells, which have the capacity to proliferate, migrate, and differentiate into endothelial cells but have not yet acquired characteristics of mature endothelial cells. The main source of adult EPCs is bone marrow, and EPCs are mobilized from bone marrow into peripheral blood (circulating EPCs) in response to certain physiological stimuli, such as, for example, tissue injury. Circulating EPCs were only recently identified in adult human blood (Asahara et al. (1997) Science 275:964-967) and subsequent studies have suggested a role for EPCs in the maintenance of endothelial integrity and function, as well as in postnatal neovascularization.

As cardiovascular disease risk factors are thought to induce vascular and cardiovascular disorders through endothelial dysfunction, and as EPCs function in tissue repair following vascular injury, reduced numbers of EPCs may be associated with endothelial dysfunction. (Quyyumi et al. (2004) Can. J. Cardiol. 20: 44B-8B; Vasa et al. (2001) Circ. Res. 89: E1-7.) Lower than normal (i.e., reduced) levels of circulating EPCs are observed in certain individuals having various cardiovascular disease risk factors, such as, for example, hypercholesterolemia, diabetes, smoking, and hypertension. (Verma et al. (2002) Circulation 105: 546-549; Vasa et al. (2001) Circ. Res. 89:e1-e7; Tepper et al. (2002) Circulation 106:2781-2786). Moreover, reduced EPC levels are an independent predictor of poor prognosis following a cardiovascular event, such as stroke. (Schmidt-Lucke et al. (2005) Circulation 111:2981-2987.) In contrast, increased circulating EPC levels correlate with improved outcomes in cardiovascular and cerebrovascular ischemia. (Sobrino et al. (2007) Stroke 38:2759-2764.) Thus, increasing circulating EPC levels may provide benefit in certain conditions associated with tissue injury.

However, EPCs are not abundant in either circulating blood or the bone marrow. In fact, the low abundance of EPCs represents one of the critical issues to overcome in the clinical application of EPCs. (Kawamoto et al. (2007) Catheterization and Cardiovascular Interventions 70:477-484.) Increased EPC levels in the clinic currently are achieved by transplantation, which involves isolating EPCs from a donor, expanding the EPCs ex vivo, and then transplanting the EPCs to the recipient. EPC transplantation is an invasive and expensive procedure, and the ex vivo manipulation of isolated EPCs poses various health risks, such as transmission of infectious agents, to both the patient and care provider.

There is thus a need for methods effective at increasing EPC levels in the blood and bone marrow which do not require costly or invasive isolation or transplantation procedures. The present invention meets this need by providing novel methods and compounds useful for increasing EPC mobilization from the bone marrow to the blood in a subject, and for increasing EPC levels in blood and bone marrow.

### SUMMARY OF THE INVENTION

The present invention also provides methods for increasing EPC levels in a subject. In one embodiment, the invention provides a method for increasing EPC levels in blood in a subject, the method comprising administering to the subject an effective amount of a compound that inhibits the activity of a hypoxia-inducible factor (HIF) prolyl hydroxylase enzyme, thereby increasing EPC levels in the blood in the subject. In another embodiment, the invention provides a method for increasing EPC levels in bone marrow in a subject, the method comprising administering to the subject an effective amount of a compound that inhibits the activity of a hypoxia-inducible factor (HIF) prolyl hydroxylase enzyme, thereby increasing EPC levels in the bone marrow in the subject.

The present invention provides methods for increasing EPC mobilization in a subject. In one embodiment, the invention provides a method for increasing mobilization of EPCs in a subject, the method comprising administering to the subject an effective amount of a compound that inhibits the activity of a hypoxia-inducible factor (HIF) prolyl hydroxylase enzyme, thereby increasing the mobilization of EPCs in the subject. In certain aspects, the mobilization of EPCs in a subject is mobilization of EPCs from the bone marrow to the blood.

In certain embodiments, a subject suitable for treatment with the present methods and compounds is a subject who has decreased or reduced EPC levels, or is at risk for having decreased or reduced EPC levels. In other embodiments, a subject having or at risk for having at least one cardiovascular disease risk factor is a subject suitable for treatment with the methods and compounds of the present invention. Cardiovascular disease risk factors include, for example, hypercholesterolemia, diabetes, smoking, hypertension, etc.

The present invention provides methods for EPC transplantation. In one embodiment, the invention provides methods for EPC transplantation in a scaffold, the method comprising: administering to a subject an effective amount of a compound that inhibits the activity of a hypoxia-inducible factor (HIF) prolyl hydroxylase enzyme; obtaining a sample of blood or bone marrow from the subject; isolating EPCs from the sample of blood or bone marrow; and seeding the EPCs in the scaffold. In another embodiment, the invention provides methods for autologous EPC transplantation, the method comprising: administering to a subject an effective amount of a compound that inhibits the activity of a hypoxia-inducible factor (HIF) prolyl hydroxylase enzyme; obtaining a sample of blood or bone marrow from the subject; isolating EPCs from the sample of blood or bone marrow; and administering the EPCs to the subject. In yet another embodiment, the invention provides methods for allogeneic EPC transplantation, the method comprising: administering to a first subject an effective amount of a compound that inhibits the activity of a hypoxia-inducible factor (HIF) prolyl hydroxylase enzyme; obtaining a sample of blood or bone marrow from the first subject; isolating EPCs from the sample of blood or bone marrow; and administering the EPCs to a second subject.

In certain embodiments, the compound used in the present methods is a structural mimetic of 2-oxoglutarate, wherein the compound inhibits the target HIF prolyl hydroxylase enzyme competitively with respect to 2-oxoglutarate and noncompetitively with respect to iron. In other embodiments, compounds of the present invention include variously substituted 3-hydroxy-pyridine-2-carbonyl-glycines, 4-hydroxy-pyridazine-3-carbonyl-glycines, 3-hydroxy-quinoline-2-carbonyl-glycines, 4-hydroxy-2-oxo-1,2-dihydro-quinoline-3-carbonyl-glycines, 4-hydroxy-2-oxo-1,2-dihydro-naphthyridine-3-carbonyl-glycines, 8-hydroxy-6-oxo-4,6-dihydro-pyridopyrazine-7-carbonyl-glycines, 4-hydroxy-isoquinoline-3-carbonyl-glycines, 4-hydroxy-cinnoline-3-carbonyl-glycines, 7-hydroxy-thienopyridine-6-carbonyl-glycines, 4-hydroxy-thienopyridine-5-carbonyl-glycines, 7-hydroxy-thiazolopyridine-6-carbonyl-glycines, 4-hydroxy-thiazolopyridine-5-carbonyl-glycines, 7-hydroxy-pyrrolopyridine-6-carbonyl-glycines, and 4-hydroxy-pyrrolopyridine-5-carbonyl-glycines. In particular embodiments, the compound is [4-Hydroxy-7-(4-methoxy-phenoxy)-isoquinoline-3-carbonyl]-amino}-acetic acid (Compound A), [(4-Hydroxy-1-methyl-7-phenoxy-isoquinoline-3-carbonyl)-amino]-acetic acid (Compound B), or [1-Cyano-6-(2,6-dimethyl-phenoxy)-4-hydroxy-isoquinoline-3-carbonyl]-amino}-acetic acid (Compound C).

In one embodiment, a compound for use in the present methods and medicaments is a pyridine-2-carboxamide, a pyridazine-3-carboxamide, a quinoline-2-carboxamide, an isoquinoline-3-carboxamide or ester thereof as described in European Patent Nos. EP0650960 and EP0650961. In another embodiment, a compound for use in the present methods and medicaments is a pyridine-2-carboxamide as described in U.S. Patent Application Publication No. 2007/0299086. In yet another embodiment, a compound for use in the present methods and medicaments is a pyridine-2-carboxamidoester, a pyridazine-3-carboxamidoester, or an isoquinoline-3-carboxamidoester as described in U.S. Pat. No. 5,658,933.

In some embodiments, a compound for use in the present methods and medicaments is a pyridine-2-carboxamide, a pyridizine-3-carboxamide, or a quinoline-2-carboxamide as described in U.S. Pat. No. 5,620,995. In another embodiment, a compound for use in the methods and medicaments of the present invention is a 3-hydroxypyridine-2-carboxamidoester as described in U.S. Pat. No. 6,020,350; a sulfonamidocarbonylpyridine-2-carboxamide as described in U.S. Pat. No. 5,607,954; or a sulfonamidocarbonyl-pyridine-2-carboxamide or a sulfonamidocarbonyl-pyridine-2-carboxamide ester as described in U.S. Pat. Nos. 5,610,172 and 5,620,996. In yet another embodiment, a compound for use in the present methods and medicaments is a quinoline-2-carboxamide as described in U.S. Pat. Nos. 5,719,164 and 5,726,305.

In other embodiments, a compound for use in the present methods and medicaments is an isoquinoline-3-carboxamide as described in U.S. Pat. Nos. 6,093,730 and 7,323,475. In another embodiment, a compound for use in the present methods and medicaments is an isoquinoline-3-carboxamide as described in U.S. Patent Application Publication No. 2007/0298104. In still another embodiment, a compound for use in the present methods and medicaments is a beta-carboline-3-carboxamide, a pyrrolo[3,2-c]pyridine-6-carboxamide, a pyrrolo[2,3-c]pyridine-5-carboxamide, a thiazolo[4,5-c]pyridine-6-carboxamide, or a thiazolo[5,4-c]pyridine-6-carboxamide as described in U.S. Patent Application Publication No. 2008/0004309.

In one embodiment, a compound for use in the present methods and medicaments is a thieno[3,2-c]pyridine-6-carboxamide or a thieno[2,3-c]pyridine-5-carboxamide as described in U.S. Patent Application Publication No. 2006/0199836. In another embodiment, a compound for use in the present methods and medicaments is a 2,4-dioxo-1,2,3,4-tetrahydro-pyrimidine-5-carboxamide or a 4-oxo-2-thioxo-1,2,3,4-tetrahydro-pyrimidine-5-carboxamide as described in International Publication No. WO 2007/150011. In yet another embodiment, a compound for use in the present methods and medicaments is a 6-oxo-1,6-dihydro-pyrimidine-5-carboxamide as described in U.S. Patent Application Publication No. 2008/0171756.

In some embodiments, a compound for use in the present methods and medicaments is a 2-oxo-1,2-dihydro-quinoline-3-carboxamide as described in International Publication No. WO 2007/038571 and U.S. Patent Application Publication No. 2007/0249605. In other embodiments, a compound for use in the present methods and medicaments is a 2-oxo-1,2-dihydro-[1,8]naphthyridine-3-carboxamide, a 2-oxo-1,2-dihydro-[1,6]naphthyridine-3-carboxamide, or a 6-oxo-5,6-dihydro-pyrido[2,3-b]pyrazine-7-carboxamide as described in International Publication Nos. WO 2007/103905, WO 2008/076425, and WO 2008/130527. In yet another embodiment, a compound for use in the present methods and medicaments is a 6-oxo-6,7-dihydro-thieno[2,3-b]pyridine-5-carboxamide, a 5-oxo-4,5-dihydro-thieno[3,2-b]pyridine-6-carboxamide, or a 6-oxo-6,7-dihydro-pyrazolo[3,4-b]pyridine-5-carboxamide as described in International Publication No. WO 2007/136990.

In one embodiment, a compound for use in the present methods and medicaments is a 3-oxo-2,3-dihydro-pyridazine-4-carboxamide as described in U.S. Patent Application Publication No. 2008/0214549. In other embodiments, a compound for use in the present methods and medicaments is a 3-oxo-3,4-dihydro-naphthalene-2-carboxamide, a 7-oxo-7,8-dihydro-quinoline-6-carboxamide, or a 7-oxo-7,8-dihydro-isoquinoline-6-carboxamide as described in International Publication No. WO 2008/076427. In another embodiment, a compound for use in the present methods and medicaments is a 3-hydroxy-1-oxo-1H-indene-2-carboxamide as described in International Publication No. WO 2008/130508.

In another embodiment, a compound for use in the present methods and medicaments is a 4-oxo-[1,10]-phenanthroline as described in U.S. Pat. Nos. 5,916,898 and 6,200,974, and International Publication No. WO 99/21860. In one aspect, a 4-oxo-[1,10]-phenanthroline is 4-oxo-1,4-dihydro-[1,10]phenanthroline-3-carboxylic acid (see, e.g., Seki et al. (1974) Chem Abstracts 81:424, No. 21).

In one embodiment, a compound for use in the present methods and medicaments is a hydrozone as described in U.S. Pat. No. 6,660,737. In other embodiments, a compound for sue in the present methods and medicaments is a dihydropyrazole or a dihydropyrozolone as described in U.S. Pat. No. 6,878,729 and International Publication No. WO 2008/049539. In another embodiment, a compound for use in the present methods and medicaments is a dipyridyl dihyropyrazones as described in International Publication No. WO 2006/114213. In other embodiments, a compound for use in the present methods and medicaments is a spiroindalone as described in International Publication No. WO 2008/144266.

In various embodiments, compounds for use in the present invention are selected from the group consisting of 2-oxoglutarate mimetics, iron chelators, and proline analogs. In preferred embodiments, the compound used in the methods and medicaments of the present invention is a 2-oxoglutarate structural mimetic. In particular embodiments, the compound used in the methods and medicaments of the present invention is a 2-oxoglutarate structural mimetic that inhibits HIF prolyl hydroxylase competitively with respect to 2-oxoglutarate and noncompetitively with respect to iron.

A compound for use in the methods and medicaments of the present invention is, in various embodiments, a cyclic carboxamide. In one aspect of the present embodiment, the cyclic carboxamide is a carbonyl glycine. In other aspects of the present embodiment, the carboxamide is replaced by a carbonyl proprionic acid. In some embodiments of the present invention, the compound used in the methods and medicaments of the present invention is a carbocyclic carboxamide.

In one embodiment, cyclic carboxamides suitable for use in the present invention are heterocyclic carboxamides. In certain embodiments, a compound of the present invention is a heterocyclic carboxamide having a heterocyclic group selected from the group consisting of: azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, thiazole, thiazolidine, thiophene, benzo[b]thiophene, morpholinyl, thiomorpholinyl (also referred to as thiamorpholinyl), piperidinyl, pyrrolidine, and tetrahydrofuranyl. In preferred embodiments, the heterocyclic group is a single ring selected from the group consisting of a pyridine, a pyridinone, a pyradizine, a pyridazinone, a pyrimidine, and a pyrimidinone ring. In other preferred embodiments, the heterocyclic group is a multiple condensed ring selected from the group consisting of an isoquinoline, an isoquinolone, a naphthyridinone, a pyrrolopyridine, a pyrrolopyridinone, a pyrozolopyridinone, a pyrrolopyridizinone, a quinoline, a quinolone, a chromenone, a thiochromenone, a thienopyridine, a thienopyridinone, a thiazolopyridine, and a thiazolopyridinone.

A particularly preferred compound of the present invention is a heterocyclic carbonyl glycine. In successive embodiments, the heterocyclic carbonyl glycine suitable for use in the present invention is a heterocyclic carbonyl glycine having a heterocyclic group that is selected from the following list: azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, thiazole, thiazolidine, thiophene, benzo[b]thiophene, morpholinyl, thiomorpholinyl (also referred to as thiamorpholinyl), piperidinyl, pyrrolidine, and tetrahydrofuranyl. In certain preferred embodiments, the heterocyclic carbonyl glycine suitable for use in the present invention is a heterocyclic carbonyl glycine having a heterocyclic group, wherein the heterocyclic group is a single ring selected from the following list: a pyridine, a pyridinone, a pyradizine, a pyridazinone, a pyrimidine, and a pyrimidinone ring. In other preferred embodiments, the heterocyclic carbonyl glycine suitable for use in the present invention is a heterocyclic carbonyl glycine having a heterocyclic group, wherein the heterocyclic group is a multiple condensed ring selected from the group consisting of an isoquinoline, an isoquinolone, a naphthyridinone, a pyrrolopyridine, a pyrrolopyridinone, a pyrozolopyridinone, a pyrrolopyridizinone, a quinoline, a quinolone, a chromenone, a thiochromenone, a thienopyridine, a thienopyridinone, a thiazolopyridine, and a thiazolopyridinone.

In one embodiment, a compound for use in the present methods and medicaments is an isoquinoline carbonyl glycine; preferably, an isoquinoline-3-carbonyl-glycine or a 4-hydroxy-isoquinoline-3-carbonyl glycine. In particular embodiments, a compound for use in the present methods and medicaments is {[4-Hydroxy-7-(4-methoxy-phenoxy)-isoquinoline-3-carbonyl]-amino}-acetic acid (Compound A); [(4-Hydroxy-1-methyl-7-phenoxy-isoquinoline-3-carbonyl)-amino]-acetic acid (Compound B); or {[1-Cyano-6-(2,6-dimethyl-phenoxy)-4-hydroxy-isoquinoline-3-carbonyl]-amino}-acetic acid (Compound C).

In other embodiments, a compound for use in the present methods and medicaments is a HIF prolyl hydroxylase inhibitor compound of Formula I:

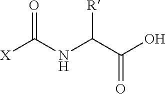

wherein X is an optionally substituted cyclic moiety and R′ is hydrogen or (C₁-C₄)-alkyl. In particular embodiments, the cyclic moiety is a heterocyclic moiety and R′ is hydrogen. Such HIF prolyl hydroyxlase inhibitors include, but are not limited to, variously substituted pyridine-2-carbonyl-glycines, pyridazine-3-carbonyl-glycines, quinoline-2-carbonyl-glycines, 2-oxo-1,2-dihydro-quinoline-3-carbonyl-glycines, 2-oxo-1,2-dihydro-naphthyridine-3-carbonyl-glycines, 6-oxo-4,6-dihydro-pyridopyrazine-7-carbonyl-glycines, isoquinoline-3-carbonyl-glycines, cinnoline-3-carbonyl-glycines, thienopyridine-6-carbonyl-glycines, thienopyridine-5-carbonyl-glycines, thiazolopyridine-6-carbonyl-glycines, thiazolopyridine-5-carbonyl-glycines, hydroxy-pyrrolopyridine-6-carbonyl-glycines, and pyrrolopyridine-5-carbonyl-glycines.

In another embodiment, a compound for use in the methods and medicaments of the present invention is a compound of Formula II:

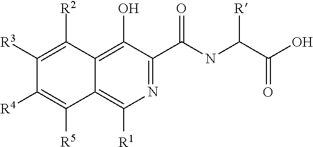

wherein:
R′ is selected from hydrogen and (C₁-C₄)-alkyl;
R¹, R², R³, R⁴ and R⁵ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-c₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl,(C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido, (C₆-C₁₂)-heteroaryl, (C₇-C₁₆)-heteroaralkyl; where an aryl or heteroaryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{2f+1-g}, —OCF₂Cl, and —OCF₂—CHFCl;
x is 0 to 3;
f is 1 to 8; and
g is 0 or 1 to (2f+1);
or a pharmaceutically acceptable salt, single stereoisomer, mixture of stereoisomers, ester, or prodrug thereof.
   In preferred embodiments, a compound of Formula II is a compound wherein:
R′ is hydrogen;
R¹ is selected from hydrogen, (C₁-C₃)-alkyl, or cyano;
R² and R⁵ are hydrogen;
R³ is hydrogen or aryloxy optionally substituted with one or two (C₁-C₃)-alkyl substituents; and
R⁴ is hydrogen or aryloxy optionally substituted with a (C₁-C₃)-alkoxy substituent.
   In other preferred embodiments, a compound of Formula II is a compound wherein:
R′ is hydrogen;
R¹ is selected from hydrogen, methyl, or cyano;
R² and R⁵ are hydrogen;
R³ is hydrogen or 2,6-dimethyl-phenoxy; and
R⁴ is selected from hydrogen, phenoxy, or 4-methoxy-phenoxy.

Pharmaceutical compositions or medicaments effective for use in any of the present methods are provided herein. In various embodiments, the compositions comprise an effective amount of a compound that inhibits the activity of a HIF prolyl hydroxylase and an acceptable carrier.

It is further contemplated that, in various embodiments, the methods of the present invention are used in combination with administration of one or more other therapeutic agents. Other therapeutic agents (subsequent or coordinate administration) for use in the present methods include EPC stimulating factors, such as statins, phosphodiesterase inhibitors, granulocyte colony-stimulating factor (G-CSF), granulocyte-macrophage colony-stimulating factor (GM-CSF), erythropoietin, vascular endothelial growth factor (VEGF), peroxisome proliferator-activated receptor-gamma (PPAR-gamma) agonists, stromal cell-derived factor-1 (SDF-1), angiopoietin-1, and estrogen.

These and other embodiments of the present invention will readily occur to those of skill in the art in light of the disclosure herein, and all such embodiments are specifically contemplated.

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a HIF-specific 2-oxoglutarate dioxygenase enzyme” may include a plurality of such enzymes; a reference to a “compound that inhibits the activity of a hypoxia-inducible factor prolyl hydroxylase enzyme” may be a reference to one or more compounds that inhibits the activity of a hypoxia-inducible factor prolyl hydroxylase enzyme, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

The section headings are used herein for organizational purposes only, and are not to be construed as in any way limiting the subject matter described herein.

### Methods

The present invention provides methods and compounds for increasing EPC levels in a subject. In particular, the present invention provides a method for increasing EPC levels in a subject, the method comprising administering to the subject an effective amount of a compound that inhibits the activity of a HIF prolyl hydroxylase enzyme, thereby increasing EPC levels in the subject. The invention also provides compounds for use in manufacturing a medicament for increasing EPC levels in a subject, wherein the compound inhibits the activity of a HIF prolyl hydroxylase enzyme.

Detection and quantitation of EPCs can be performed by any measure known to those skilled in the art. For example, detection of EPCs in a sample obtained from a human subject is performed by flow cytometry as previously described. (Vasa et al. (2001) Circ. Res. 89:e1-e7). EPCs can be identified by flow cytometry, as well as other methods, by the presence of one of three characteristic and identifying cell surface markers: the hematopoietic progenitor cell marker CD34; the immature hematopoietic marker CD133; and the endothelial cell receptor KDR (other aliases for KDR include VEGFR-2 and Flk-1). (Lambiase et al. (2005) Circulation 109:2986-2992.) Recent studies concluded that a CD34+/KDR+ double-positive phenotype on a cell is the most appropriate for the identification of EPCs. (Ben-Shoshan et al. (2007) Pharmacology & Therapeutics 115:25-36.) Therefore, in certain embodiments, the methods and compounds of the present invention are useful for increasing EPC levels, wherein the EPCs are positive for expression of both CD34 and KDR cell-surface markers (i.e., double positive; CD34+/KDR+). In other embodiments, methods and compounds for increasing EPC levels are provided, wherein the EPCs have at least one cell surface marker selected from the group consisting of CD34, KDR, VEGFR-2, Flk-1, and CD133.

Adult EPCs derive primarily from bone marrow. In response to certain physiological stimuli, such as, for example, tissue injury, EPCs are mobilized from bone marrow into peripheral blood and into circulation. Therefore, it is an object of the present invention to provide methods and compounds that increase EPC mobilization. In one embodiment, the methods and compounds of the present invention are useful for increasing mobilization of EPCs in a subject, the method comprising administering to the subject an effective amount of a compound that inhibits the activity of a hypoxia-inducible factor (HIF) prolyl hydroxylase enzyme, thereby increasing mobilization of EPCs in the subject. In certain aspects, the mobilization of EPCs is mobilization from bone marrow to blood.

In another embodiment, the methods and compounds of the present invention are useful for increasing EPC levels in blood in a subject, the method comprising administering to the subject an effective amount of a compound that inhibits the activity of a hypoxia-inducible factor (HIF) prolyl hydroxylase enzyme, thereby increasing EPC levels in the in blood in the subject. In yet another embodiment, the methods and compounds of the present invention are useful for increasing EPC levels in bone marrow of a subject, the method comprising administering to the subject an effective amount of a compound that inhibits the activity of a hypoxia-inducible factor (HIF) prolyl hydroxylase enzyme, thereby increasing EPC levels in bone marrow in the subject.

It is further contemplated that, in various embodiments, the methods and compounds of the present invention are used in combination with administration of one or more other therapeutic agents. Other therapeutic agents (subsequent or coordinate administration) for use in the present methods include EPC stimulating factors, such as statins, phosphodiesterase inhibitors, G-CSF, GM-CSF, erythropoietin, VEGF, PPAR-gamma agonists, SDF-1, angiopoietin-1, and estrogen.

The present methods and compounds are useful for and effective at increasing EPC levels in a subject, wherein the EPCs are functional EPCs, i.e., EPCs that are able to differentiate into mature endothelial cells. Various cell culture methods are available to identify functional EPCs. (See, e.g., Asahara et al. (1997) Science 275:964-967; and Hill et al. (2003) New Engl J Med 348:593-600.) In these culture methods, adult peripheral blood mononuclear cells are plated on fibronectin-coated dishes. After a 48 h pre-plating step to deplete the sample of adherent macrophages and adherent mature endothelial cells (ECs), the non-adherent cells are removed and re-plated on fresh fibronectin-coated dishes, and cultured. Discrete colonies emerge in 5-9 days, comprised of round cells (centrally) with spindle-shaped cells sprouting at the periphery. Such colonies are referred to as colony-forming unit-endothelial cells (CFU-ECs).

The present invention provides methods and agents useful for EPC transplantation in scaffolds. EPC transplantation in biocompatible and biodegradable scaffolds has been utilized for the repairing or regenerating skin, heart, nerve, liver, pancreas, cartilage, and bone tissue using various biological and synthetic materials. (See, e.g., Derval et al. (2008) Eur J Cardiothorac Surg. 34:248-54.) EPC transplantation on such scaffolds can be accomplished by, for example, harvesting and purifying EPCs from the blood or bone marrow of a donor subject; seeding the purified EPCs in a scaffold; and then subsequently implanting the seeded scaffold to a recipient subject. (See, e.g., Wu et al. (2004) Am J Physiol Heart Circ Physiol. 287:H480-H487.) Methods and agents of the present invention increase EPC levels in the blood and bone marrow of subjects. (See, e.g. Example 1.) Therefore, use of methods of the present invention to increase EPC levels in a subject for subsequent EPC transplantation in scaffolds is specifically provided.

The present invention further provides methods and agents useful for EPC transplantation procedures. Transplantation of EPCs has been carried out in human subjects. (See, e.g., Assmus et al. (2002) Circulation 106:3009-3017.) Generally, EPCs are harvested and purified from the subject's bone marrow or blood, expanded ex-vivo, and subsequently administered to the same subject (i.e. autologous) or another subject (i.e. allogeneic). Methods and agents of the present invention increase EPC levels in the blood and bone marrow of subjects. (See, e.g. Example 1.) Thus, use of methods of the present invention to increase EPC levels in a subject for subsequent EPC transplantation is specifically provided.

### Subjects

The present invention relates to methods for increasing EPC mobilization and increasing EPC levels in a subject by administering to the subject an effective amount of a compound that inhibits the activity of a hypoxia-inducible factor prolyl hydroxylase enzyme.

The invention is applicable to a variety of different organisms, including, for example, vertebrates, large animals, and primates. In a preferred embodiment, the subject is a mammalian subject, and in a most preferred embodiment, the subject is a human subject. However, although medical applications with humans are clearly foreseen, veterinary applications are also envisaged herein.

The methods of the present invention are particularly suitable for subjects who would benefit from increased EPC mobilization or increased EPC levels, such as, for example, having or at risk for having an ischemic disease or tissue injury. In some aspects, a suitable subject is a subject that has low or reduced EPC levels. In other aspects, a suitable subject is a subject that has normal EPC levels. In yet other aspects, a suitable subject is a subject that has high or increased EPC levels.

Whether a subject has low (reduced), normal, or high (increased) EPC levels is determined by any measure accepted and utilized by those skilled in the art. For example, low, normal, and high EPC levels in peripheral blood samples obtained from humans have been described. (Xiao et al. (2007) PLoS ONE 2:e975.) In human blood, a low or reduced level of EPCs is about 17-295 EPCs per 10⁶ peripheral blood mononuclear cells (PBMNCs); a normal level of EPCs is about 296-859 EPCs per 10⁶ PBMNCs; a high or increased level of EPCs is about 860-4,768 per 10⁶ PBMNCs.

In one embodiment, the methods and compounds of the present invention are applied to increase EPC levels in a subject having low or reduced EPC levels. In another embodiment, the methods and compounds of the present invention are applied to a subject having normal EPC levels. In yet another embodiment, the methods and compounds of the present invention are applied to a subject with high or increased EPC levels.

It is specifically contemplated that methods and compounds of the present invention are applied to older subjects, as an age-related decline in bone marrow EPCs has been observed. (Hill et al. (2003) N Engl J Med 348:593-600.)

### Compounds

Compounds for use in the methods or medicaments provided herein are inhibitors of HIF prolyl hydroxylase enzymes. The term “HIF prolyl hydroxylase,” as used herein, refers to any enzyme that is capable of hydroxylating a proline residue within an alpha subunit of HIF. Such HIF prolyl hydroxylases include protein members of the EGL-9 (EGLN) 2-oxoglutarate- and iron-dependent dioxygenase family described by Taylor (2001) Gene 275:125-132 and characterized by Aravind and Koonin (2001) Genome Biol 2:RESEARCH0007; Epstein et al. (2001) Cell 107:43-54; and Bruick and McKnight (2001) Science 294:1337-1340. Examples of HIF prolyl hydroxylases include human SM-20 (EGLN1) (GenBank Accession No. AAG33965; Dupuy et al. (2000) Genomics 69:348-54), EGLN2 isoform 1 (GenBank Accession No. CAC42510; Taylor, supra), EGLN2 isoform 2 (GenBank Accession No. NP_{—}060025), and EGLN3 (GenBank Accession No. CAC42511); mouse EGLN1 (GenBank Accession No. CAC42515), EGLN2 (GenBank Accession No. CAC42511), and EGLN3 (SM-20) (GenBank Accession No. CAC42517); and rat SM-20 (GenBank Accession No. AAA19321). Additionally, HIF prolyl hydroxylase may include Caenorhabditis elegans EGL-9 (GenBank Accession No. AAD56365) and Drosophila melanogaster CG1114 gene product (GenBank Accession No. AAF52050). The term “HIF prolyl hydroxylase” also includes any active fragment of the foregoing full-length proteins.

A compound that inhibits the activity of a HIF prolyl hydroxylase enzyme is any compound that reduces or otherwise modulates the activity of at least one HIF prolyl hydroxylase enzyme. A compound may additionally show inhibitory activity toward one or more other 2-oxoglutarate- and iron-dependent dioxygenase enzymes, e.g. factor inhibiting HIF (FIH; GenBank Accession No. AAL27308), procollagen prolyl 4-hydroxylase (cP4H), etc. In particular embodiments, compounds used in the present methods and medicaments provided herein are structural mimetics of 2-oxoglutarate, wherein the compound inhibits the target HIF prolyl hydroxylase enzyme competitively with respect to 2-oxoglutarate and noncompetitively with respect to iron. Examples of compounds that may be used in the methods and medicaments provided herein can be found, e.g., in Majamaa et al. (1984) Eur. J. Biochem. 138:239-245; Majamaa et al. (1985) Biochem. J. 229:127-133; Kivirikko, and Myllyharju (1998) Matrix Biol. 16:357-368; Bickel et al. (1998) Hepatology 28:404-411; Friedman et al. (2000) Proc. Natl. Acad. Sci. USA 97:4736-4741; Franklin (1991) Biochem. Soc. Trans. 19):812-815; and Franklin et al. (2001) Biochem. J. 353:333-338. Additionally, compounds that inhibit HIF hydroxylase enzyme activity have been described in, e.g., International Publication Nos. WO 03/049686, WO 02/074981, WO 03/080566, WO 2004/108681, WO 2006/094292, WO 2007/038571, WO 2007/090068, WO 2007/070359, WO 2007/103905, and WO 2007/115315.

Examples of additional compounds that may be used in the methods and medicaments provided herein include, but are not limited to, variously substituted 3-hydroxy-pyridine-2-carbonyl-glycines, 4-hydroxy-pyridazine-3-carbonyl-glycines, 3-hydroxy-quinoline-2-carbonyl-glycines, 4-hydroxy-2-oxo-1,2-dihydro-quinoline-3-carbonyl-glycines, 4-hydroxy-2-oxo-1,2-dihydro-naphthyridine-3-carbonyl-glycines, 8-hydroxy-6-oxo-4,6-dihydro-pyridopyrazine-7-carbonyl-glycines, 4-hydroxy-isoquinoline-3-carbonyl-glycines, 4-hydroxy-cinnoline-3-carbonyl-glycines, 7-hydroxy-thienopyridine-6-carbonyl-glycines, 4-hydroxy-thienopyridine-5-carbonyl-glycines, 7-hydroxy-thiazolopyridine-6-carbonyl-glycines, 4-hydroxy-thiazolopyridine-5-carbonyl-glycines, 7-hydroxy-pyrrolopyridine-6-carbonyl-glycines, and 4-hydroxy-pyrrolopyridine-5-carbonyl-glycines. In particular embodiments, the compound is [4-Hydroxy-7-(4-methoxy-phenoxy)-isoquinoline-3-carbonyl]-amino}-acetic acid (Compound A), [(4-Hydroxy-1-methyl-7-phenoxy-isoquinoline-3-carbonyl)-amino]-acetic acid (Compound B), or [1-Cyano-6-(2,6-dimethyl-phenoxy)-4-hydroxy-isoquinoline-3-carbonyl]-amino}-acetic acid (Compound C).

Compounds for use in the present invention are compounds that inhibit HIF prolyl hydroxylase activity. A compound that inhibits HIF prolyl hydroxylase activity is any compound that reduces or otherwise inhibits the activity of at least one HIF prolyl hydroxylase enzyme. Various compounds that inhibit HIF prolyl hydroxylase have been identified and are suitable for use in the methods and medicaments as claimed in the present invention.

Exemplary pyridine-2-carboxamides, pyridazine-3-carboxamides, quinoline-2-carboxamides, isoquinoline-3-carboxamides and esters thereof are described in European Patent Nos. EP0650960 and EP0650961. All compounds listed in EP0650960 and EP0650961, in particular, those listed in the compound claims and the final products of the working examples, are hereby incorporated into the present application by reference herein. Additional pyridine-2-carboxamides are described in U.S. Patent Application Publication No. 2007/0299086. All compounds listed in U.S. Patent Application Publication No. 2007/0299086, in particular, those listed in the compound claims and the final products of the working examples, are hereby incorporated into the present application by reference herein. Additionally, exemplary pyridine-2-carboxamidoesters, pyridazine-3-carboxamidoesters, and isoquinoline-3-carboxamidoesters are described in U.S. Pat. No. 5,658,933. All pyridine-2-carboxamidoesters, pyridazine-3-carboxamidoesters, and quinoline-2-carboxamidesters listed in U.S. Pat. No. 5,658,933, in particular, those listed in the compound claims and the final products of the working examples, are hereby incorporated into the present application by reference herein.

Additional pyridine-2-carboxamides, pyridizine-3-carboxamides, and quinoline-2-carboxamides are described in U.S. Pat. No. 5,620,995. All compounds listed in U.S. Pat. No. 5,620,995, in particular, those listed in the compound claims and the final products of the working examples, are hereby incorporated into the present application by reference herein. Exemplary 3-hydroxypyridine-2-carboxamidoesters are described in U.S. Pat. No. 6,020,350; sulfonamidocarbonylpyridine-2-carboxamides are described in U.S. Pat. No. 5,607,954; and sulfonamidocarbonyl-pyridine-2-carboxamides and sulfonamidocarbonyl-pyridine-2-carboxamide esters are described in U.S. Pat. Nos. 5,610,172 and 5,620,996. All compounds listed in these patents, in particular, those compounds listed in the compound claims and the final products of the working examples, are hereby incorporated into the present application by reference herein.

Exemplary quinoline-2-carboxamides are described in U.S. Pat. Nos. 5,719,164 and 5,726,305. All compounds listed in the foregoing patents, in particular, those listed in the compound claims and the final products of the working examples, are hereby incorporated into the present application by reference herein.

Exemplary isoquinoline-3-carboxamides are described in U.S. Pat. Nos. 6,093,730 and 7,323,475. All compounds listed in U.S. Pat. Nos. 6,093,730 and 7,323,475, in particular, those listed in the compound claims and the final products of the working examples, are hereby incorporated into the present application by reference herein. Particularly exemplary embodiments of isoquinoline-3-carboxamides are described in U.S. Patent Application Publication No. 2007/0298104. All compounds listed in U.S. Patent Application Publication No. 2007/0298104, in particular, those listed in the compound claims and the final products of the working examples, are hereby incorporated into the present application by reference herein.

Exemplary beta-carboline-3-carboxamides, pyrrolo[3,2-c]pyridine-6-carboxamides, pyrrolo[2,3-c]pyridine-5-carboxamides, thiazolo[4,5-c]pyridine-6-carboxamides, and thiazolo[5,4-c]pyridine-6-carboxamides are described in U.S. Patent Application Publication No. 2008/0004309. All compounds listed in U.S. Patent Application Publication No. 2008/0004309, in particular, those listed in the compound claims and the final products of the working examples, are hereby incorporated into the present application by reference herein.

Exemplary thieno[3,2-c]pyridine-6-carboxamide and thieno[2,3-c]pyridine-5-carboxamides are described in U.S. Patent Application Publication No. 2006/0199836. All compounds listed in U.S. Patent Application Publication No. 2006/0199836, in particular, those listed in the compound claims and the final products of the working examples, are hereby incorporated into the present application by reference herein.

Exemplary 2,4-dioxo-1,2,3,4-tetrahydro-pyrimidine-5-carboxamides and 4-oxo-2-thioxo-1,2,3,4-tetrahydro-pyrimidine-5-carboxamides are described in International Publication No. WO 2007/150011. All compounds listed in the foregoing publication, in particular, those listed in the compound claims and the final products of the working examples, are hereby incorporated into the present application by reference herein. Exemplary 6-oxo-1,6-dihydro-pyrimidine-5-carboxamides are described in U.S. Patent Application Publication No. 2008/0171756. All compounds listed in U.S. Patent Application Publication No. 2008/0171756, in particular, those listed in the compound claims and the final products of the working examples, are hereby incorporated into the present application by reference herein.

Exemplary 2-oxo-1,2-dihydro-quinoline-3-carboxamides are described in International Publication No. WO 2007/038571 and U.S. Patent Application Publication No. 2007/0249605. All compounds listed in the foregoing publications, in particular, those listed in the compound claims and the final products of the working examples, are hereby incorporated into the present application by reference herein.

Exemplary 2-oxo-1,2-dihydro-[1,8]naphthyridine-3-carboxamides, 2-oxo-1,2-dihydro-[1,6]naphthyridine-3-carboxamides, and 6-oxo-5,6-dihydro-pyrido[2,3-b]pyrazine-7-carboxamides are described in International Publication Nos. WO 2007/103905, WO 2008/076425, and WO 2008/130527. All compounds listed in the foregoing publications, in particular, those listed in the compound claims and the final products of the working examples, are hereby incorporated into the present application by reference herein.

Exemplary 6-oxo-6,7-dihydro-thieno[2,3-b]pyridine-5-carboxamides, 5-oxo-4,5-dihydro-thieno[3,2-b]pyridine-6-carboxamides, 6-oxo-6,7-dihydro-pyrazolo[3,4-b]pyridine-5-carboxamides are described in International Publication No. WO 2007/136990. All compounds listed in the foregoing publications, in particular, those listed in the compound claims and the final products of the working examples, are hereby incorporated into the present application by reference herein.

Exemplary 3-oxo-2,3-dihydro-pyridazine-4-carboxamides are described in U.S. Patent Application Publication No. 2008/0214549. All compounds listed in U.S. Patent Application Publication No. 2008/0214549, in particular, those listed in the compound claims and the final products of the working examples, are hereby incorporated into the present application by reference herein.

Exemplary 3-oxo-3,4-dihydro-naphthalene-2-carboxamides, 7-oxo-7,8-dihydro-quinoline-6-carboxamides, and 7-oxo-7,8-dihydro-isoquinoline-6-carboxamides are described in International Publication No. WO 2008/076427. All compounds listed in the foregoing publication, in particular, those listed in the compound claims and the final products of the working examples, are hereby incorporated into the present application by reference herein.

Exemplary 3-hydroxy-1-oxo-1H-indene-2-carboxamides are described in International Publication No. WO 2008/130508. All compounds listed in International Publication No. WO 2008/130508, in particular, those listed in the compound claims and the final products of the working examples, are hereby incorporated into the present application by reference herein.

Exemplary 4-oxo-[1,10]-phenanthrolines are described in U.S. Pat. Nos. 5,916,898 and 6,200,974, and International Publication No. WO 99/21860. All compounds listed in the foregoing patents and publication, in particular, those listed in the compound claims and the final products of the working examples, are hereby incorporated into the present application by reference herein. An exemplary 4-oxo-[1,10]-phenanthroline is 4-oxo-1,4-dihydro-[1,10]phenanthroline-3-carboxylic acid (see, e.g., Seki et al. (1974) Chem Abstracts 81:424, No. 21).

Exemplary hydrozones are described in U.S. Pat. No. 6,660,737. All compounds listed in U.S. Pat. No. 6,660,737, in particular, those listed in the compound claims and the final products of the working examples, are hereby incorporated into the present application by reference herein.

Exemplary dihydropyrazoles and dihydropyrozolones are described in U.S. Pat. No. 6,878,729 and International Publication No. WO 2008/049539, respectively. All compounds listed in U.S. Pat. No. 6,878,729, in particular, those listed in the compound claims and the final products of the working examples, are hereby incorporated into the present application by reference herein. Exemplary dipyridyl dihyropyrazones are described in International Publication No. WO 2006/114213. All compounds listed in International Publication No. WO 2006/114213, in particular, those listed in the compound claims and the final products of the working examples, are hereby incorporated into the present application by reference herein.

Exemplary spiroindalones are described in International Publication No. WO 2008/144266. All compounds listed in International Publication No. WO 2008/144266, in particular, those listed in the compound claims and the final products of the working examples, are hereby incorporated into the present application by reference herein.

Additional HIF prolyl hydroxylase inhibitors known to those of skill in the art are described in Dao et al. (2009, Anal Biochem 384(2):213-23), Frohn et al. (2008, Bioorg Med Chem Lett 18(18):5023-6), and Tegley et al. (2008, Bioorg Med Chem Lett 18(14):3925-8). All compounds listed in the foregoing publications are hereby incorporated into the present application by reference herein.

In various embodiments, compounds suitable for use in the present invention are selected from the group consisting of 2-oxoglutarate mimetics, iron chelators, and proline analogs. In preferred embodiments, the compound is a 2-oxoglutarate structural mimetic.

2-oxoglutarate structural mimetics suitable for use in the claimed methods include structural mimetics of 2-oxoglutarate that inhibit HIF prolyl hydroxylase activity competitively with respect to 2-oxoglutarate. In preferred embodiments, the compound is a 2-oxoglutarate structural mimetic that inhibits HIF prolyl hydroxylase competitively with respect to 2-oxoglutarate and noncompetitively with respect to iron.

A compound of the present invention is, in various embodiments, a cyclic carboxamide. In some embodiments, the cyclic carboxamide is a carbonyl glycine. In other embodiments, the carboxamide is replaced by a carbonyl proprionic acid. In some embodiments of the present invention, the compound of the present invention is a carbocyclic carboxamide.

Preferred cyclic carboxamides suitable for use in the present invention are heterocyclic carboxamides. Such heterocyclic carboxamide compounds include heterocyclic carboxamides previously identified as inhibitors of HIF prolyl hydroxylase activity, and known and available to those of skill in the art. In certain embodiments, a compound of the present invention is a heterocyclic carboxamide having a heterocyclic group selected from the group consisting of: azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, thiazole, thiazolidine, thiophene, benzo[b]thiophene, morpholinyl, thiomorpholinyl (also referred to as thiamorpholinyl), piperidinyl, pyrrolidine, and tetrahydrofuranyl. In preferred embodiments, the heterocyclic group is a single ring selected from the group consisting of a pyridine, a pyridinone, a pyradizine, a pyridazinone, a pyrimidine, and a pyrimidinone ring. In other preferred embodiments, the heterocyclic group is a multiple condensed ring selected from the group consisting of an isoquinoline, an isoquinolone, a naphthyridinone, a pyrrolopyridine, a pyrrolopyridinone, a pyrozolopyridinone, a pyrrolopyridizinone, a quinoline, a quinolone, a chromenone, a thiochromenone, a thienopyridine, a thienopyridinone, a thiazolopyridine, and a thiazolopyridinone.

A particularly preferred heterocyclic carboxamide of the present invention is a heterocyclic carbonyl glycine. Such preferred heterocyclic carbonyl glycines include those represented by Formula I, infra. In successive embodiments, the heterocyclic carbonyl glycine suitable for use in the present invention is a heterocyclic carbonyl glycine having a heterocyclic group that is selected from the following list: azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, thiazole, thiazolidine, thiophene, benzo[b]thiophene, morpholinyl, thiomorpholinyl (also referred to as thiamorpholinyl), piperidinyl, pyrrolidine, and tetrahydrofuranyl. In certain preferred embodiments, the heterocyclic carbonyl glycine suitable for use in the present invention is a heterocyclic carbonyl glycine having a heterocyclic group, wherein the heterocyclic group is a single ring selected from the following list: a pyridine, a pyridinone, a pyradizine, a pyridazinone, a pyrimidine, and a pyrimidinone ring. In other preferred embodiments, the heterocyclic carbonyl glycine suitable for use in the present invention is a heterocyclic carbonyl glycine having a heterocyclic group, wherein the heterocyclic group is a multiple condensed ring selected from the group consisting of an isoquinoline, an isoquinolone, a naphthyridinone, a pyrrolopyridine, a pyrrolopyridinone, a pyrozolopyridinone, a pyrrolopyridizinone, a quinoline, a quinolone, a chromenone, a thiochromenone, a thienopyridine, a thienopyridinone, a thiazolopyridine, and a thiazolopyridinone.

Particular heterocyclic carbonyl glycines most suitable for use in the claimed methods include isoquinoline carbonyl glycines; preferably, isoquinoline-3-carbonyl-glycines. Further preferred isoquinoline-3-carbonyl glycines include 4-hydroxy-isoquinoline-3-carbonyl glycines. Exemplary such compounds include {[4-Hydroxy-7-(4-methoxy-phenoxy)-isoquinoline-3-carbonyl]-amino}-acetic acid (Compound A); [(4-Hydroxy-1-methyl-7-phenoxy-isoquinoline-3-carbonyl)-amino]-acetic acid (Compound B); and {[1-Cyano-6-(2,6-dimethyl-phenoxy)-4-hydroxy-isoquinoline-3-carbonyl]-amino}-acetic acid (Compound C); and other compounds represented by Formula II, infra.

As discussed, supra, in one embodiment, a compound of the present invention is a HIF prolyl hydroxylase inhibitor compound of Formula I:

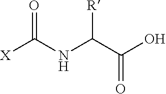

wherein X is an optionally substituted cyclic moiety and R′ is hydrogen or (C₁-C₄)-alkyl. In particular embodiments, the cyclic moiety is a heterocyclic moiety and R′ is hydrogen. Such HIF prolyl hydroyxlase inhibitors include, but are not limited to, variously substituted pyridine-2-carbonyl-glycines, pyridazine-3-carbonyl-glycines, quinoline-2-carbonyl-glycines, 2-oxo-1,2-dihydro-quinoline-3-carbonyl-glycines, 2-oxo-1,2-dihydro-naphthyridine-3-carbonyl-glycines, 6-oxo-4,6-dihydro-pyridopyrazine-7-carbonyl-glycines, isoquinoline-3-carbonyl-glycines, cinnoline-3-carbonyl-glycines, thienopyridine-6-carbonyl-glycines, thienopyridine-5-carbonyl-glycines, thiazolopyridine-6-carbonyl-glycines, thiazolopyridine-5-carbonyl-glycines, hydroxy-pyrrolopyridine-6-carbonyl-glycines, and pyrrolopyridine-5-carbonyl-glycines.

In another embodiment, a compound of the present invention is a compound of Formula II:

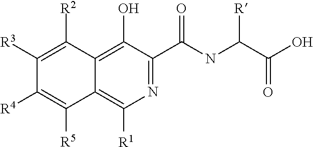

wherein:
R′ is selected from hydrogen and (C₁-C₄)-alkyl;
R¹, R², R³, R⁴ and R⁵ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-c₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido, (C₆-C₁₂)-heteroaryl, (C₇-C₁₆)-heteroaralkyl; where an aryl or heteroaryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H(_{2f+1-g)}F_{g}, —OCF₂Cl, and —OCF₂—CHFCl;
x is 0 to 3;
f is 1 to 8; and
g is 0 or 1 to (2f+1);
or a pharmaceutically acceptable salt, single stereoisomer, mixture of stereoisomers, ester, or prodrug thereof.

In preferred embodiments, a compound of Formula II is a compound wherein:
R′ is hydrogen;
R¹ is selected from hydrogen, (C₁-C₃)-alkyl, or cyano;
R² and R⁵ are hydrogen;
R³ is hydrogen or aryloxy optionally substituted with one or two (C₁-C₃)-alkyl substituents; and
R⁴ is hydrogen or aryloxy optionally substituted with a (C₁-C₃)-alkoxy substituent.

In other preferred embodiments, a compound of Formula II is a compound wherein:
R′ is hydrogen;
R¹ is selected from hydrogen, methyl, or cyano;
R² and R⁵ are hydrogen;
R³ is hydrogen or 2,6-dimethyl-phenoxy; and
R⁴ is selected from hydrogen, phenoxy, or 4-methoxy-phenoxy.

The terms “hydroxy” or “hydroxyl” refer to the group —OH.

The term “halo” or “halogen” refers to fluoro, chloro, bromo, and iodo.

The term “cyano” refers to the group —CN.

The term “nitro” refers to the group —NO₂.

The term “carboxyl” refers to —COOH or salts thereof.

The term “alkyl” refers to saturated monovalent hydrocarbyl groups having from 1 to 10 carbon atoms; more particularly, from 1 to 5 carbon atoms; and, even more particularly, 1 to 3 carbon atoms. This term is exemplified by groups such as methyl, ethyl, n-propyl, iso-propyl, n-butyl, t-butyl, n-pentyl, and the like.

The term “cycloalkyl” refers to a saturated or an unsaturated, but nonaromatic, cyclic alkyl groups of from 3 to 10, 3 to 8, or 3 to 6 carbon atoms having single or multiple cyclic rings including, by way of example, adamantyl, cyclopropyl, cyclobutyl, cyclopentyl, cyclooctyl, cyclohexenyl, and the like.

The term “cycloalkoxy” refers to an —O-cycloalkyl group.

The term “aryl” refers to a monovalent aromatic carbocyclic group of from 6 to 14 carbon atoms having a single ring (e.g., phenyl) or multiple condensed rings (e.g., naphthyl or anthryl), which condensed rings may or may not be aromatic (e.g., 2-benzoxazolinone, 2H-1,4-benzoxazin-3(4H)-one-7-yl, and the like) provided that the point of attachment is the aryl group. Preferred aryls include phenyl and naphthyl.

The terms “heterocyclic” or “heterocyclyl” refer to a saturated or unsaturated ring system having a single ring or multiple condensed rings, from 1 to 10 carbon atoms, and from 1 to 4 hetero atoms selected from the group consisting of nitrogen, sulfur, or oxygen within the ring.

The term “heteroaryl” refers to an aromatic heterocyclic group of from 1 to 15 carbon atoms, preferably from 1 to 10 carbon atoms, and 1 to 4 heteroatoms within the ring selected from the group consisting of oxygen, nitrogen, and sulfur. Such heteroaryl groups can have a single ring (e.g., pyridinyl, furyl, or thienyl) or multiple condensed rings (e.g., indolizinyl or benzothienyl), which condensed rings may or may not be aromatic provided the point of attachment is through a ring containing the heteroatom and that ring is aromatic. The nitrogen can optionally be oxidized to provide for the N-oxide, and/or the sulfur ring atoms can optionally be oxidized to provide for the sulfoxide and sulfone derivatives.

Examples of heterocycles and heteroaryls include, but are not limited to, azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

The term “alkenyl” refers to a vinyl unsaturated monovalent hydrocarbyl group having from 2 to 6, preferably from 2 to 4, carbon atoms, and having at least 1, preferably from 1 to 2, sites of vinyl (>C═C<) unsaturation. Such groups are exemplified by vinyl (ethen-1-yl), allyl, but-3-enyl, and the like.

The term “alkynyl” refers to acetylinic unsaturated monovalent hydrocarbyl groups having from 2 to 6, preferably from 2 to 3, carbon atoms and having at least 1, preferably from 1 to 2, sites of acetylenic (—C≡C—) unsaturation. This group is exemplified by ethyn-1-yl, propyn-1-yl, propyn-2-yl, and the like.

The term “alkoxy” refers to the group “alkyl-O—,” which includes, by way of example, methoxy, ethoxy, n-propoxy, iso-propoxy, n-butoxy, t-butoxy, sec-butoxy, n-pentoxy, and the like.

The term “alkenyloxy” refers to the group “alkenyl-O—.”

The term “alkynyloxy” refers to the group “alkynyl-O—.”

The term “aryloxy” refers to the group aryl-O— that includes, by way of example, phenoxy, naphthoxy, and the like.

The term “aralkyloxy” refers to the group aralkyl-O— that includes, by way of example, benzyloxy, and the like.

The term “carbonyl” refers to C═O.

The term “carbonyloxy” refers to —C(═O)O—.

The terms “aminoacyl” or “amide”, or the prefixes “carbamoyl” or “carboxamide,” refer to the group —C(O)NR^{q}R^{q} where each R^{q} is independently selected from the group consisting of hydrogen, alkyl, alkenyl, alkynyl, aryl, cycloalkyl, heteroaryl, and heterocyclic; or where each R^{q} is joined to form together with the nitrogen atom a heterocyclic wherein alkyl, alkenyl, alkynyl, cycloalkyl, aryl, heteroaryl, and heterocyclic are as defined herein.

The term “amino” refers to the group —NH₂.

The terms “thio” or “mercapto” refer to the group —SH.

The terms “alkylsulfanyl,” “alkylthio,” or “thioether” refer to the groups —S-alkyl where alkyl is as defined above.

The term “sulfinyl” refers to the group —S(O)—.

The term “sulfonyl” refers to the group —S(O)₂—.

The term “heterocyclyloxy” refers to the group —O-heterocyclic.

The term “cycloalkylene” refers to divalent cycloalkyl groups as defined above. The terms “cycloalkylthio” or “cycloalkylsulfanyl” refer to the groups —S-cycloalkyl where cycloalkyl is as defined herein.

The terms “arylthio” or “arylsulfanyl” refer to the group —S-aryl, where aryl is as defined herein.

The terms “heteroarylthio” or “heteroarylsulfanyl” refer to the group —S-heteroaryl, where heteroaryl is as defined herein.

The terms “heterocyclicthio” or “heterocyclicsulfanyl” refer to the group —S-heterocyclic, where heterocyclic is as defined herein.

The term “alkyl alcohol” refers to the group “alkyl-OH”. “Alkyl alcohol” is meant to include methanol, ethanol, 2-propanol, 2-butanol, butanol, etc.

The term “acyl” refers to the groups H—C(O)—, alkyl-C(O)—, alkenyl-C(O)—, alkynyl-C(O)—, cycloalkyl-C(O)—, aryl-C(O)—, heteroaryl-C(O)—, and heterocyclic-C(O)—, provided that a nitrogen atom of the heterocyclic is not bound to the —C(O)— group, wherein alkyl, alkenyl, alkynyl, cycloalkyl, aryl, heteroaryl, and heterocyclic are as defined herein.

The term “acyloxy” refers to the groups alkyl-C(O)O—, alkenyl-C(O)O—, alkynyl-C(O)O—, aryl-C(O)O—, cycloalkyl-C(O)O—, heteroaryl-C(O)O—, and heterocyclic-C(O)O—, wherein alkyl, alkenyl, alkynyl, cycloalkyl, aryl, heteroaryl, and heterocyclic are as defined herein.

The term “alkenyl” refers to a vinyl unsaturated monovalent hydrocarbyl group having from 2 to 6 carbon atoms, and preferably 2 to 4 carbon atoms, and having at least 1, and preferably from 1 to 2 sites of vinyl (>C═C<) unsaturation. Such groups are exemplified by vinyl (ethen-1-yl), allyl, but-3-enyl and the like.

The term “alkynyl” refers to acetylinic unsaturated monovalent hydrocarbyl groups having from 2 to 6, preferably from 2 to 3, carbon atoms and having at least 1, preferably from 1 to 2, sites of acetylenic (—C≡C—) unsaturation. This group is exemplified by ethyn-1-yl, propyn-1-yl, propyn-2-yl, and the like.

The term “acylamino” refers to the groups —NR^{t}C(O)-alkyl, —NR^{t}C(O)cycloalkyl, —NR^{t}C(O)alkenyl, —NR^{t}C(O)alkynyl, —NR^{t}C(O)aryl, —NR^{t}C(O)heteroaryl, and —NR^{t}C(O)heterocyclic where R^{t} is hydrogen or alkyl, and wherein alkyl, alkenyl, alkynyl, cycloalkyl, aryl, heteroaryl, and heterocyclic are defined herein.

The term “carbonyloxyamino” refers to the groups —NR^{u}C(O)O-alkyl, —NR^{u}C(O)O-alkenyl, —NR^{u}C(O)O-alkynyl, —NR^{u}C(O)O-cycloalkyl, —NR^{u}C(O)O-aryl, —NR^{u}C(O)O-heteroaryl, and —NR^{u}C(O)O-heterocyclic, where R^{u} is hydrogen or alkyl and wherein alkyl, alkenyl, alkynyl, cycloalkyl, aryl, heteroaryl, and heterocyclic are as defined herein.

The term “oxycarbonylamino” refers to the groups —NR^{u}C(O)O-alkyl, —NR^{u}C(O)O-alkenyl, —NR^{u}C(O)O-alkynyl, —NR^{u}C(O)O-cycloalkyl, —NR^{u}C(O)O-aryl, —NR^{u}C(O)O-heteroaryl, and —NR^{u}C(O)O-heterocyclic, where R^{u} is hydrogen or alkyl, and wherein alkyl, alkenyl, alkynyl, cycloalkyl, aryl, heteroaryl, and heterocyclic are as defined herein.

The term “oxythiocarbonylamino” refers to the groups —NR^{u}C(S)O-alkyl, —NR^{u}C(S)O-alkenyl, —NR^{u}C(S)O-alkynyl, —NR^{u}C(S)O-cycloalkyl, —NR^{u}C(S)O-aryl, —NR^{u}C(S)O-heteroaryl, and —NR^{u}C(S)O-heterocyclic, where R^{u} is hydrogen or alkyl, and wherein alkyl, alkenyl, alkynyl, cycloalkyl, aryl, heteroaryl, and heterocyclic are as defined herein.

The term “aminocarbonyloxy” or the prefix “carbamoyloxy” refer to the groups —OC(O)NR^{v}R^{v} where each R^{v} is independently selected from the group consisting of hydrogen, alkyl, alkenyl, alkynyl, cycloalkyl, aryl, heteroaryl, and heterocyclic; or where each R^{v} is joined to form, together with the nitrogen atom, a heterocyclic, and wherein alkyl, alkenyl, alkynyl, cycloalkyl, aryl, heteroaryl, substituted heteroaryl, and heterocyclic are as defined herein.

The term “aminocarbonylamino” refers to the group —NR^{w}C(O)N(R^{w})₂ where each R^{W} is independently selected from the group consisting of hydrogen and alkyl.

The term “aminothiocarbonylamino” refers to the group —NR^{w}C(S)N(R^{w})₂ where each R^{w} is independently selected from the group consisting of hydrogen and alkyl.

The term “aryloxyaryl” refers to the group -aryl-O-aryl.

The term “carboxyl ester” refers to the groups —C(O)O-alkyl, —C(O)O-alkenyl, —C(O)O-alkynyl, —C(O)O-cycloalkyl, —C(O)O-aryl, —C(O)O-substituted aryl, —C(O)O-heteroaryl, —C(O)O-substituted heeteroaryl, —C(O)O-heterocyclic, and —C(O)O-substituted heterocyclic.

The term “cycloalkylene” refers to divalent cycloalkyl groups as defined above.

The term “heteroaryloxy” refers to the group —O-heteroaryl.

The term “sulfonyl” refers to the group —S(O)₂—, and may be included in the groups —S(O)₂H, —SO₂-alkyl, —SO₂-alkenyl, —SO₂-alkynyl, —SO₂-cycloalkyl, —SO₂-cycloalkenyl, —SO₂-aryl, —SO₂-substituted aryl, —SO₂-heteroaryl, and —SO₂-heterocyclic, wherein alkyl, alkenyl, alkynyl, cycloalkyl, cycloalkenyl, aryl, heteroaryl, and heterocyclic are as defined herein.

The term “heterocyclyloxy” refers to the group —O-heterocyclic.

The terms “arylthio” or “arylsulfanyl” refer to the group —S-aryl.

The terms “heteroarylthio” or “heteroarylsulfanyl” refer to the group —S-heteroaryl.

The terms “heterocyclicthio” or “heterocyclicsulfanyl” refer to the group —S-heterocyclic.

Conjugated terms refer to a linear arrangement of the separate substituents as each separate term is defined herein. For example, the term “aralkyl” refers to an aryl-alkyl group and includes, by way of example, benzyl; the term “aralkylcarbamoyl” refers to an aryl-alkyl-carbomoyl substituent wherein each term is as defined herein, etc.

It is understood that in all substituted and conjugated groups as defined herein, polymers arrived at by defining substituents with further substituents to themselves (e.g., aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three.

Similarly, it is understood that the above definitions are not intended to include impermissible substitution patterns (e.g., methyl substituted with 5 fluoro groups or a hydroxyl group alpha to ethenylic or acetylenic unsaturation). Such impermissible substitution patterns are well known to the skilled artisan.

The term “pharmaceutically acceptable salt” refers to pharmaceutically acceptable salts of a compound, which salts are derived from a variety of organic and inorganic counter ions well known in the art, and include, by way of example only, sodium, potassium, calcium, magnesium, ammonium, tetraalkylammonium, and the like; and, when the molecule contains a basic functionality, salts of organic or inorganic acids, such as hydrochloride, hydrobromide, tartrate, mesylate, acetate, maleate, oxalate, and the like.

The terms “stereoisomer” or “stereoisomers” refer to compounds that differ in the chirality of one or more stereocenters. Stereoisomers include enantiomers (compounds are non-superimposable mirror images) and diastereomers (compounds having more than one stereogenic center that are non-mirror images of each other and wherein one or more stereogenic center differs between the two stereoisomers). The compounds of the invention can be present as a mixture of stereoisomers or as a single stereoisomer.

The term “tautomer” refers to alternate forms of a compound that differ in the position of a proton, such as enol, keto, and imine enamine tautomers, or the tautomeric forms of heteroaryl groups containing a ring atom attached to both a ring NH moiety and a ring ═N moiety such as pyrazoles, imidazoles, benzimidazoles, triazoles, and tetrazoles.

The term “prodrug,” as used herein, refers to compounds that include chemical groups which, in vivo, can be converted into the carboxylate group and/or can be split off from the amide N-atom and/or can be split off from the R′ atom to provide for the active drug, a pharmaceutically acceptable salt thereof, or a biologically active metabolite thereof. Suitable groups are well known in the art and particularly include: for the carboxylic acid moiety, a prodrug selected from, e.g., esters including, but not limited to, those derived from alkyl alcohols, substituted alkyl alcohols, hydroxy substituted aryls and heteroaryls and the like; amides, particularly amides derived from amines of the Formula HNR²⁰⁰R²¹⁰ where R²⁰⁰ and R²¹⁰ are independently hydrogen, alkyl, substituted alkyl, aryl, substituted aryl, and the like; hydroxymethyl, aldehyde and derivatives thereof. The term “ester” refers to compounds that include the group —COOR where R is alkyl, substituted alkyl, alkoxy, or substituted alkoxy.

The term “excipient” as used herein means an inert or inactive substance used in the production of pharmaceutical products or other tablets, including without limitation any substance used as a binder, disintegrant, coating, compression/encapsulation aid, cream or lotion, lubricant, parenteral, sweetener or flavoring, suspending/gelling agent, or wet granulation agent. Binders include, e.g., carbopol, povidone, xanthan gum, etc.; coatings include, e.g., cellulose acetate phthalate, ethylcellulose, gellan gum, maltodextrin, etc.; compression/encapsulation aids include, e.g., calcium carbonate, dextrose, fructose dc, honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams and lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; parenterals include, e.g., mannitol, povidone, etc.; plasticizers include, e.g., dibutyl sebacate, polyvinylacetate phthalate, etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

### Methods for Identifying Compounds

Methods for identifying compounds of the invention are also provided. Assays for hydroxylase activity are standard in the art. Such assays can directly or indirectly measure hydroxylase activity. For example, an assay can measure hydroxylated residues, e.g., proline, asparagine, etc., present in the enzyme substrate, e.g., a target protein, a synthetic peptide mimetic, or a fragment thereof. (See, e.g., Palmerini et al. (1985) J Chromatogr 339:285-292.) A reduction in hydroxylated residue, e.g., proline or asparagine, in the presence of a compound is indicative of a compound that inhibits hydroxylase activity. Alternatively, assays can measure other products of the hydroxylation reaction, e.g., formation of succinate from 2-oxoglutarate. (See, e.g., Cunliffe et al. (1986) Biochem J 240:617-619.) Kaule and Gunzler (1990; Anal Biochem 184:291-297) describe an exemplary procedure that measures production of succinate from 2-oxoglutarate.

Procedures such as those described above can be used to identify compounds that modulate HIF hydroxylase activity. Target protein may include HIFα or a fragment thereof, e.g., HIF(556-575). Enzyme may include, e.g., HIF prolyl hydroxylase or active fragments thereof (see, e.g., GenBank Accession No. AAG33965, etc.) or HIF asparaginyl hydroxylase active fragments thereof (see, e.g., GenBank Accession No. AAL27308, etc.), obtained from any source. Enzyme may also be present in a crude cell lysate or in a partially purified form. For example, procedures that measure HIF hydroxylase activity are described in Ivan et al. (2001, Science 292:464-468; and 2002, Proc Natl Acad Sci USA 99:13459-13464) and Hirsila et al. (2003, J Biol Chem 278:30772-30780); additional methods are described in International Publication No. WO 03/049686. Measuring and comparing enzyme activity in the absence and presence of the compound will identify compounds that inhibit hydroxylation of HIFα.

### Pharmaceutical Formulations and Routes of Administration

The compositions of the present invention can be delivered directly or in pharmaceutical compositions containing excipients, as is well known in the art. The present methods of treatment involve administration of an effective amount of a compound of the present invention to a subject in need, wherein the subject has reduced or is at risk for having reduced EPC levels, or wherein the subject would benefit by having increased EPC mobilization or increased EPC levels.

An effective amount, e.g., dose, of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Hardman, Limbird, and Gilman, eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictates the type of formulation and the route of administration to be used, as well as whether local or systemic delivery would be preferred.

In preferred embodiments, the compounds of the present invention are administered orally. For example, in certain embodiments, the invention provides for oral administration of Compound A [4-Hydroxy-7-(4-methoxy-phenoxy)-isoquinoline-3-carbonyl]-amino}-acetic acid, Compound B [(4-Hydroxy-1-methyl-7-phenoxy-isoquinoline-3-carbonyl)-amino]-acetic acid, or Compound C [1-Cyano-6-(2,6-dimethyl-phenoxy)-4-hydroxy-isoquinoline-3-carbonyl]-amino}-acetic acid.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro)-emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

A therapeutically effective dose or amount of a compound, agent, or drug of the present invention refers to an amount or dose of the compound, agent, or drug that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the compound or pharmaceutical composition that will elicit the biological or medical response of a tissue, system, animal, or human that is being sought by the researcher, veterinarian, medical doctor, or other clinician, e.g., treatment of cancer, including induction of anti-tumor effects, etc.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

In some embodiments of the present invention, effective doses for compounds of the invention include doses of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, and 30 mg/kg, respectively.

In additional embodiments, effective treatment regimes for compounds of the invention include administration two or three times weekly.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing. Such modifications fall within the scope of the appended claims.

### Example 1

### Increased Levels of Endothelial Progenitor Cells in Mouse

To examine the effect of compounds and methods of the present invention on endothelial progenitor cell (EPC) mobilization and EPC levels, the following studies were performed. Male Swiss-Webster mice were administered various compounds of the present invention via oral gavage using a ball-tipped gavage needle. Animals treated by oral gavage received a 10 ml/kg volume of either 0.5% carboxymethyl cellulose (CMC) with 0.1% Polysorbate 80 (vehicle control) or various doses (10-100 mg/kg) of a compound of the present invention in 0.5% CMC with 0.1% Polysorbate 80. Animals were dosed once daily for 3 or 4 days. Six hours after the final dosing, 200 μl blood samples were collected for detection of EPCs by FACS analysis as previously described. (Vasa et al. (2001) Circ Res 89:e1-e7.) Briefly, mononuclear cells were isolated from whole blood samples by erythrocyte depletion using red blood cell lysis buffer (eBioscience) according to the manufacturer's instructions. The remaining cells were incubated for one hour with monoclonal antibodies directed against mouse CD34 and mouse KDR, cell surface markers for EPCs (BD Pharmingen). Following incubation with the anti-CD34 and anti-KDR monoclonal antibodies, the samples were washed three times with sort buffer (PBS, 1% fetal bovine serum) and resuspended in sort buffer containing 50 μg/ml of propidium iodide. Samples were then analyzed for the presence and quantitation of EPCs by flow cytometry (FACS Calibur, Becton Dickinson). The cytometer was set to acquire 10 000 events. Two or three samples obtained from each animal were analyzed and the average recorded. EPCs were determined as double positive KDR+/CD34+ cells.

As shown below in Table 1, compounds of the present invention were effective at increasing both EPC levels in blood and at increasing the percent of EPCs in blood. These results indicated that methods and compounds of the present invention are effective at mobilizing EPCs and increasing EPC levels in blood. These results further showed that EPC levels in the blood increased following oral administration of various compounds of the present invention.

TABLE 1

| | | | | | Percent Blood | |
|---|---|---|---|---|---|---|
| | Dose | Days | Blood EPCs | Fold Increase | EPCs | Fold Increase |
| Compound | (mg/kg) | Dosed | (cells/μl) | Over Control | (% gated cells) | Over Control |
| A | 60 | 3 | 24.7 | 2.4 | 2.6 | 2.0 |
| A | 100 | 4 | 309.0 | 8.4 | 3.2 | 4.7 |
| B | 60 | 3 | 10.4 | 1.0 | 1.0 | 0.8 |
| B | 100 | 4 | 304.0 | 8.2 | 3.0 | 4.3 |
| C | 20 | 3 | 151.9 | 4.2 | 1.3 | 1.8 |
| C | 60 | 3 | 195.1 | 5.4 | 1.4 | 1.9 |
| C | 20 | 4 | 125.9 | 2.5 | 0.7 | 1.0 |
| C | 60 | 4 | 240.0 | 4.7 | 1.0 | 1.3 |

### Example 2

### Increased Levels of Endothelial Progenitor Cells in Bone Marrow

To examine the effect of compounds and methods of the present invention on EPC levels in bone marrow, the following studies were performed. Male Swiss-Webster mice were administered various compounds of the present invention via oral gavage using a ball-tipped gavage needle. Animals treated by oral gavage received a 10 ml/kg volume of either 0.5% carboxymethyl cellulose (CMC) with 0.1% Polysorbate 80 (vehicle control) or various doses (10-100 mg/kg) of a compound of the present invention in 0.5% CMC with 0.1% Polysorbate 80. Animals were dosed once daily for 3 or 4 days. Six hours after the final dosing, bone marrow samples were taken from one tibia of each animal and suspended in buffer (PBS with 1% fetal bovine serum). Bone marrow suspensions were then filtered through nylon filters to remove stromal cells. Following filtration, the remaining cells were counted manually using a hemocytometer. Detection and quantitation of EPCs was performed by FACS analysis as described above in Example 1.

As shown below in Table 2, compounds of the present invention were effective at increasing the percent of EPCs in the bone marrow. These results indicated that methods and compounds of the present invention are effective at increasing EPC levels in bone marrow.

TABLE 2

| Com- | Dose | Days | Percent Bone Marrow EPCs | Fold Increase |
|---|---|---|---|---|
| pound | (mg/kg) | Dosed | (% gated cells) | Over Control |
| A | 60 | 3 | 6.7 | 2.0 |
| A | 100 | 4 | 14.1 | 1.8 |
| B | 60 | 3 | 9.0 | 2.7 |
| B | 100 | 4 | 16.1 | 2.1 |
| C | 20 | 3 | 2.2 | 2.2 |
| C | 60 | 3 | 3.2 | 3.2 |
| C | 20 | 4 | 0.6 | 0.9 |
| C | 60 | 4 | 0.9 | 1.4 |

### Example 3

### Increased Levels of Colony-Forming Unit-Endothelial Cells Ex Vivo

To examine the effect of compounds and methods of the present invention on increasing EPCs, wherein the EPCs are functional EPCs, i.e., EPCs that are able to differentiate into mature endothelial cells, the following studies were performed in which the frequency of endothelial colony-forming cells was evaluated ex vivo. In one series of experiments, male Swiss-Webster mice were administered various compounds of the present invention via oral gavage using a ball-tipped gavage needle. Animals treated by oral gavage received a 10 ml/kg volume of either 0.5% carboxymethyl cellulose (CMC) with 0.1% Polysorbate 80 (vehicle control) or various doses (10-100 mg/kg) of a compound of the present invention in 0.5% CMC with 0.1% Polysorbate 80. Animals were dosed once daily for 3 or 4 days. Six hours after the final dosing, 200 μl blood samples were collected for analysis of EPC function and differentiation using an EPC colony forming assay.

EPC colony forming assays were carried out as previously described. (Murphy et al. (2007) Arterioscler Thromb Vasc Biol 27:936-942.) Briefly, mononuclear cells were isolated from whole blood samples by erythrocyte depletion using red blood cell lysis buffer (eBioscience) according to the manufacturer's instructions. The remaining cells (50,000 cells/well) were plated on fibronectin coated 24-well dishes (BD, BioSciences Discovery Labware) and incubated in endothelial growth media (EGM-2 media, Cambrex) for 12 days. At the end the incubation period, EPC colonies were counted.

As shown below in Table 3, compounds of the present invention were effective at increasing the frequency of colony-forming unit-endothelial cells (CFU-EC). These results showed that methods and compounds of the present invention are effective at increasing EPC levels in the blood. Taken together, these results further showed that the increased blood EPCs achieved using compounds and methods of the present invention are functional and can differentiate into endothelial cells.

TABLE 3

| Com- | Dose | Days | Blood CFU-EC | Fold Increase |
|---|---|---|---|---|
| pound | (mg/kg) | Dosed | (colonies/50,000 cells) | Over Control |
| A | 60 | 3 | 16.8 | 8.4 |
| A | 100 | 4 | 17.8 | 3.63 |
| B | 60 | 3 | 12.4 | 6.2 |
| B | 100 | 4 | 9.3 | 1.9 |
| C | 20 | 3 | 7.7 | 4.3 |
| C | 60 | 3 | 7.4 | 4.14 |
| C | 20 | 4 | 5.6 | 4.3 |
| C | 60 | 4 | 6.6 | 5.1 |

In another series of experiments, male Swiss-Webster mice were administered various compounds of the present invention via oral gavage using a ball-tipped gavage needle. Animals treated by oral gavage received a 10 ml/kg volume of either 0.5% carboxymethyl cellulose (CMC) with 0.1% Polysorbate 80 (vehicle control) or various doses (10-100 mg/kg) of a compound of the present invention in 0.5% CMC with 0.1% Polysorbate 80. Animals were dosed once daily for 3 or 4 days. Six hours after the final dosing, bone marrow samples were taken from one tibia of each animal. Bone marrow samples were then analyzed for EPC function and differentiation using an EPC colony forming assay as described above.

As shown below in Table 4, compounds of the present invention were effective at increasing the frequency of colony-forming unit-endothelial cells (CFU-EC). These results showed that methods and compounds of the present invention are effective at increasing EPC levels in the bone marrow. These results further showed that the increased bone marrow EPCs achieved using compounds and methods of the present invention are functional and can differentiate into endothelial cells.

TABLE 4

| Com- | Dose | Days | Bone Marrow CFU-EC | Fold Increase |
|---|---|---|---|---|
| pound | (mg/kg) | Dosed | (colonies/50,000 cells) | Over Control |
| A | 60 | 3 | 29.0 | 3.0 |
| A | 100 | 4 | 24.8 | 3.2 |
| B | 60 | 3 | 22.0 | 2.2 |
| B | 100 | 4 | 22.4 | 2.9 |
| C | 20 | 3 | 25.7 | 5.1 |
| C | 60 | 3 | 28.0 | 5.6 |
| C | 20 | 4 | 26.0 | 1.9 |
| C | 60 | 4 | 61.0 | 4.5 |

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

1. A method for increasing endothelial progenitor cell levels in a subject in need, the method comprising administering to the subject an effective amount of a compound that inhibits the activity of a hypoxia-inducible factor (HIF) prolyl hydroxylase enzyme, thereby increasing endothelial progenitor cell levels in the subject.

2. A method for increasing mobilization of endothelial progenitor cells in a subject in need, the method comprising administering to the subject an effective amount of a compound that inhibits the activity of a hypoxia-inducible factor (HIF) prolyl hydroxylase enzyme, thereby increasing mobilization of endothelial progenitor cells in the subject.

3. (canceled)

4. (canceled)

5. The method of claim 1, wherein the endothelial progenitor cell levels are increased in blood in the subject.

6. The method of claim 1, wherein the endothelial progenitor cell levels are increased in bone marrow in the subject.

7. The method of claim 2, wherein the mobilization of endothelial progenitor cells in the subject is mobilization of endothelial progenitor cells from bone marrow to blood in the subject.
